(19) United States
(12) Patent Application Publication
(10) Pub. No.: US 2010/0267958 A1
(43) Pub. Date: Oct. 21, 2010

(54) METHOD FOR ISOLATION AND PURIFICATION OF MONTELUKAST

(76) Inventors: Ales Halama, Pardubice (CZ); Josef Jirman, Praha (CZ); Hana Petrickova, Praha (CZ)

Correspondence Address: Pearl Cohen Zedek Latzer, LLP, 1500 Broadway, 12th Floor, New York, NY, 10036, US

(21) Appl. No.: 12/667,147

(22) PCT Filed: Jul. 8, 2008
(86) PCT No.: PCT/CZ08/00081

§ 371 Date: May 24, 2010

(30) Foreign Application Priority Data
Jul. 9, 2007 (CZ) PV 2007-455

(51) Int. Cl. C07D 215/14

(52) U.S. Cl. 546/174

(57) ABSTRACT

A method of isolation of Montelukast of formula I from reaction mixtures, comprising conversion of the crude sub-stance to well-crystallizing salts with primary amines in the environment of at least one organic solvent and acetonitrile, followed by re-crystallization of these salts with simultaneous removal of chemical impurities and use of the chemically pure salts of Montelukast with primary amines for direct transformation to the pharmaceutically useful amorphous form of Montelukast sodium of formula II.

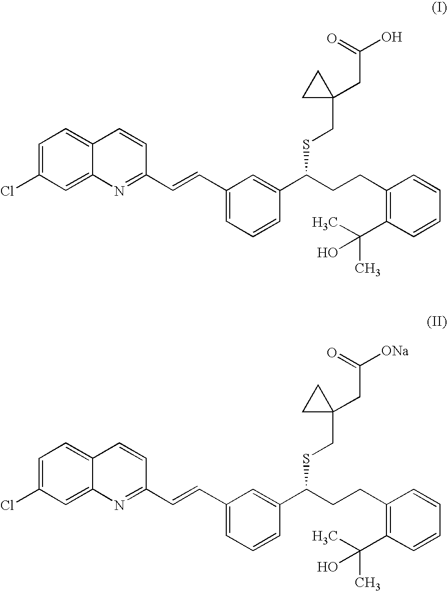

### TECHNICAL FIELD

The invention relates to a new method of isolation and purification of Montelukast of formula I, i.e. a substance that is used for the preparation of a drug for treatment of asthma and allergies.

### BACKGROUND ART

Montelukast, chemically [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid (I), is a well-known anti-asthmatic and anti-allergic drug. It is mainly the sodium salt of Montelukast described with formula (II) that is used therapeutically.

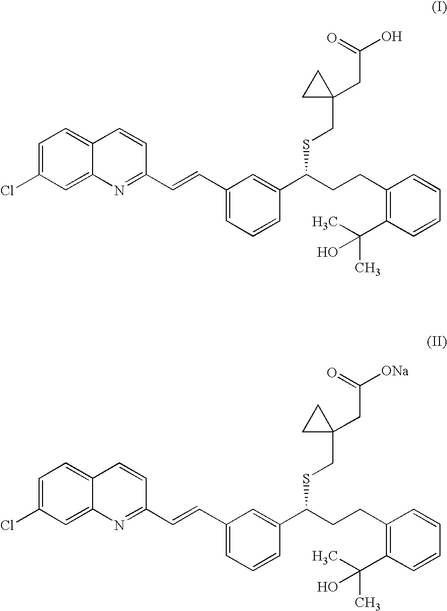

Preparation of Montelukast Sodium can be Divided into Three Partial Processes. First of all these are processes comprising many alternatives of chemical synthesis that end in the crude product stage. This is the case of solid forms or solutions of crude Montelukast acid or a crude salt of Montelukast with a metal, most frequently sodium or lithium. Another very significant process besides the chemical synthesis is the process of isolation of Montelukast from reaction mixtures and subsequent processes of chemical purification that make it possible to obtain the product in a pharmaceutically acceptable quality. For the purpose of isolation and chemical purification salts of Montelukast with amines and Montelukast acid in the solid state are used. Thirdly, there are processes that produce the pharmaceutically suitable form of Montelukast, which is its sodium salt, especially its amorphous form. Processes leading to both the amorphous form of sodium Montelukast and crystalline or semi-crystalline forms have been described.

The first solution of chemical synthesis of Montelukast (I) was described in the patent no. EP 0 480 717 B1 and subsequently in the specialized literature (M. Labele, Bioorg. Med. Chem. Lett. 5 (3), 283-288 (1995)). Other possibilities of chemical synthesis of Montelukast (I) are described in the following patents: EP 0 480 717 B1, EP 0 737 186 B1, US 2005/0234241 A1, WO 2005/105751 A1, US 2005/0107612 A1, WO 2005/105749 A2 and WO 2005/105750 A1.

For the process of isolation and purification of crude Montelukast salts of Montelukast with some amines or Montelukast acid in the solid state have been used so far. Out of salts of Montelukast salts with dicyclohexylamine (EP 0 737 186 B1, WO 2004/108679 A1), tert-butylamine (US 2005/0107612 A1, WO 2006/043846 A1), ethylphenylamine (US 2005/0107612 A1), iso-propylamine (WO 2007/005965 A1) and di-n-propylamine (WO 2007/005965 A1) have been described. Solid forms of Montelukast acid, both crystalline and amorphous, have been described in a number of patent applications: WO 2005/040123, WO 2005/073194 A2, WO 2005/074893 A1, WO 2005/074893 A1, WO 2004/108679 A1, WO 2005/074935 A1. In practice the method of purification of crude Montelukast (I) via its salts with secondary amines, especially with dicyclohexylamine, is mainly used.

The sodium salt of Montelukast, its preparation and various forms, amorphous or crystalline are described in a number of patents or patent applications, e.g. amorphous Montelukast sodium is dealt with in EP 0 737 186 B1, WO 03/066598 A1, WO 2004/108679 A1, WO 2005/074893 A1, WO 2006/054317 A1 and WO 2007/005965. Crystalline polymorphs of Montelukast sodium are described in WO 2004/091618 A1 and WO 2005/075427 A2.

Processes of isolation and purification of Montelukast are of an extraordinary economic significance as they make it possible to obtain a substance useful for pharmaceutical purposes. These processes are used to remove both impurities primarily soluble in water and those primarily soluble in other solvents. Organic impurities have their origin in the chemical instability of Montelukast as well as in the instability of raw materials used for its synthesis, or these may be residues of used volatile substances, mainly solvents. Literature describes increased sensitivity of the target substance to oxygen (see equation (1)) while as the main product of oxidation of Montelukast (I) the substance of chemical formula (V) is described (E. D. Nelson, J. Pharm. Sci. 95, 1527-1539 (2006), C. Dufresne, J. Org. Chem. 1996, 61, 8518-8525)). Introduction of these and other impurities into the product is extremely undesirable.

For this reason processes are carried out that produce the target substance with the exclusion of oxygen, i.e. under the protection of inert gas atmosphere (e.g. nitrogen in accordance with EP 0 737 186 B1).

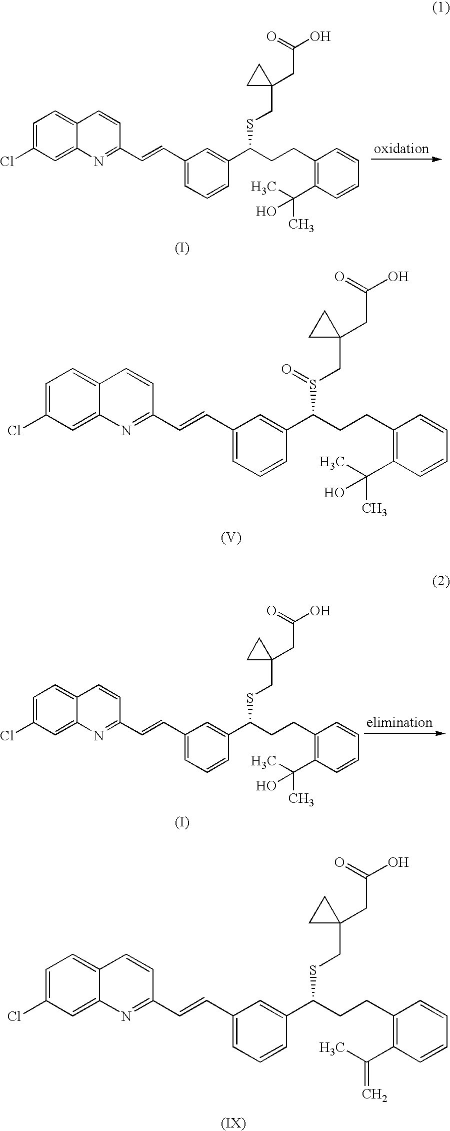

Another impurity of Montelukast described in literature (WO 2007/005965 A1) coming from instability of the target substance is the compound described with chemical formula (IX), which is formed from Montelukast through elimination of a water molecule in accordance with equation (2).

Another source of chemical contamination of Montelukast is the instability of the commonly used starting material described with formula (IV). This substance is subject to three undesired reactions—hydrolysis, elimination and cyclization with the formation of impurities described with formulae (VI-VIII), see Scheme 1 (J. O. Egekeze, Anal.Chem. 1995, 67, 2292-2295).

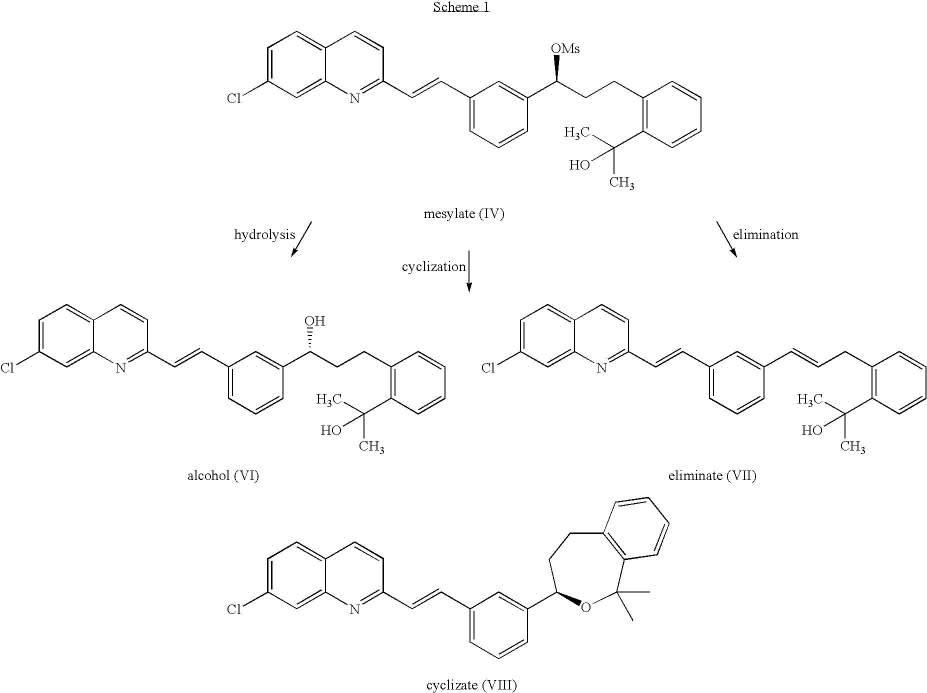

The solution presented by us represents a new, highly efficient and convenient method of isolation of crude Montelukast from reaction mixtures, especially in the form of its salts with primary amines, and subsequent crystallization of these salts with simultaneous removal of undesired impurities. Salts of Montelukast with primary amines obtained in accordance with our method can be advantageously directly transformed into the pharmaceutically useful amorphous form of Montelukast sodium (II).

### DISCLOSURE OF INVENTION

The invention deals with a new method of isolation of Montelukast prepared in the form of a solution of its salt with an alkali metal in accordance with Scheme 2, subsequent conversion of the Montelukast salt solution to a solution of Montelukast acid and further isolation of crystalline salts of Montelukast with primary amines. The invention further deals with a preferable method of removal of chemical impurities through crystallization of Montelukast salts with primary amines and a new method of preparation of the amorphous form of Montelukast sodium using direct transformation of Montelukast salts with primary amines in accordance with the procedure shown in Scheme 2.

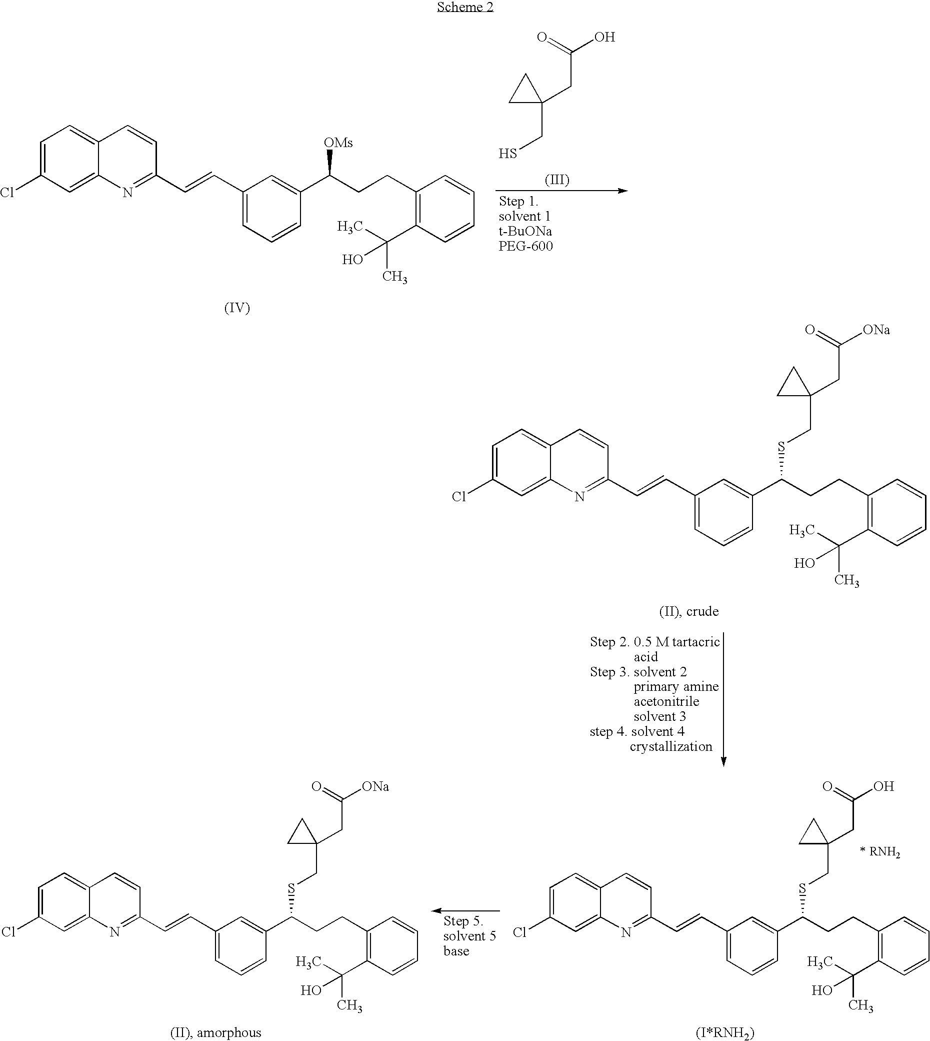

The process invented by us and described in Scheme 3 achieves Montelukast sodium in five steps.
Step 1—represents chemical synthesis of Montelukast, especially the key substitution reaction creating a link between the atoms of carbon and sulfur, forming the principal skeleton of the target molecule. The output of this step is a reaction mixture that contains a salt of Montelukast with an alkali metal and other undesired constituents.
Step 2—represents treatment of the reaction mixture that essentially consists in conversion of the solution of crude sodium or another metal salt of Montelukast to a solution of Montelukast acid while impurities soluble in water are separated at the same time. The output of this step is a solution of Montelukast acid in an organic solvent together with undesired components that are not soluble in water.
Step 3—represents isolation of a salt of Montelukast with a primary amine with the use of at least one solvent and acetonitrile as the component preventing separation of the product in a form that cannot be technologically isolated. The output of this step is a crude crystalline salt of Montelukast with a primary amine.
Step 4—represents crystallization of the salt of Montelukast with a primary amine from at least one organic solvent, while this way undesired admixtures that are primarily soluble in the used solvent are removed. The output of this step is a chemically pure crystalline salt of Montelukast with a primary amine.
Step 5—represents the process of direct transformation of Montelukast salts with primary amines to the pharmaceutically useful amorphous sodium salt. The output of this step is an active pharmaceutical substance useful in the preparation of medicaments for asthma and allergies.

The present invention essentially consists in processes concerning isolation and chemical purification of Montelukast, i.e. steps 2 to 4. It also includes the process of preparation of the amorphous form of Montelukast sodium, which is based on use of salts of Montelukast with primary amines, step 5. A very important aspect that is inevitable and original in our method of isolation of Montelukast salts is the use of acetonitrile in stage 3. Acetonitrile specifically and beneficially prevents adhesion of crystals to the walls of the crystallization vessel or to the stirrer. Thus, our presented method can be applied in the production scale with substantial advantages without the risk of excessive losses of the product that would otherwise remain stuck on the production equipment. The modes of carrying out the individual steps is described in a more detailed way below.

Step 1—Chemical Synthesis

In preparation of Montelukast by the process of present invention, 2-(2-(3(S)-(3-(2-(7-chloro-2-quinolinyl)ethenyl)phenyl)-3-methanesulfonyloxypropyl)phenyl)-2-propanol described with formula (IV), containing the methanesulfonyl group as the leaving group, was used as the first starting material. Preparation of (IV) is carried out, for example, in the manner described in patents nos. EP 0 737 186 B1, WO 2005/105751 A1 in accordance with equation (3).

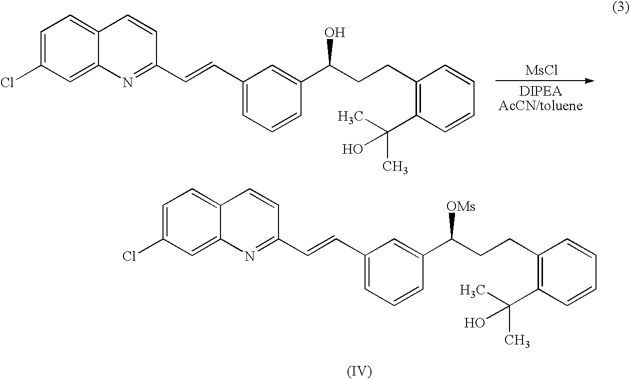

We used [1-(mercaptomethyl)cyclopropyl]-acetic acid (III) as the second starting material of the process of the present invention, which is, by the action of two equivalents of the (t-BuONa) base, directly converted in situ to the corresponding salt, which is the active form of the agent. This conversion is described with equation (4).

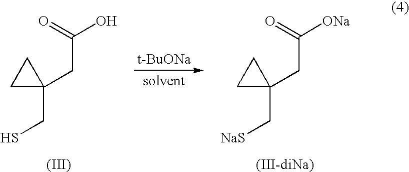

As the reaction environment, organic solvents, especially aromatic hydrocarbons and ethers or their mixtures in any proportions, are used. For example, a mixture of toluene and tetrahydrofuran is suitable. One can preferably add a component increasing selectivity of the reaction, which increases solubility and reactivity of the used nucleophilic reagent, i.e. (III-diNa). This way, the undesired impact of competing reactions on the resulting composition of the reaction mixture is limited. As the component increasing selectivity of the reaction one can use a polyether, e.g. polyethyleneglycol.

The reactions leading to the target substance (I) were carried out in the method of the present invention in such a manner that at first the carboxylic acid of formula (III) was mixed with a base (e.g. t-BuONa) and the component increasing selectivity of the reaction (e.g. PEG-600) in an inert solvent and under an inert gas atmosphere. The resulting mixture was cooled below −10° C. and then a solution of the starting substance (IV) in a suitable organic solvent was added dropwise. Further, the reaction mixture was stirred under an inert atmosphere at the temperature of −10 to 25° C. for several hours and samples were gradually taken for determination of the conversion and selectivity of the substitution reaction. The result of this step is a solution of the crude salt of Montelukast with an alkali metal. According to HPLC analyses this solution usually contained 80 to 85% of this salt.

Step 2—Treatment of the Reaction Mixture

The reaction mixture obtained by the procedure of step 1 was concentrated in vacuo. Mainly the more volatile tetrahydrofuran was evaporated. The residue was washed with a solution of an acid with water. After drying (Na₂SO₄) and filtration the filtrate was concentrated in vacuo. By this treatment of the reaction mixture the solution of the crude sodium or other salt of Montelukast is converted to a solution of Montelukast acid. At the same time undesired components soluble in water (e.g. sodium methanesulfonate, PEG-600, t-butylalcohol) are efficiently removed. In this step efficient removal of impurities that are primarily soluble in organic solvents does not occur.

Step 3—Isolation of the Salt of Montelukast with a Primary Amine

The concentrated residue obtained by the procedure of step 2 was diluted with an aromatic hydrocarbon to the required volume and then acetonitrile, a primary amine and subsequently a non-polar solvent, preferably heptane or hexane, were added. The mixture was then stirred until separation of the product. The salt of Montelukast with the primary amine was isolated in the solid state with the yield of 65-75%, HPLC purity >90%. In this step, partial removal of impurities primarily soluble in organic solvents occurs. A very substantial and advantageous aspect of our method of isolation of the salt of Montelukast with a primary amine is the use of acetonitrile as the component preventing separation of the product in a technologically non-isolable form. Thus, the addition of acetonitrile allows crystallization from the whole volume without excessive sticking of crystals to the walls of the crystallization vessel or the stirrer. When acetonitrile was not used, the addition of a non-polar solvent resulted in separation of the product in the form of oil that turned into solid mass solidifying on the walls of the vessel and the stirrer during stirring of the crystallization mixture. The product separated in this manner is not suitable for processing in the production scale. In model cases the process of preparation of salts of Montelukast with various amines, including primary, secondary and tertiary amines was tested, see example 12. The highest yields were achieved for n-propylamine (95%) and iso-propylamine (94%). The worst yield was achieved in the case of salts of Montelukast with tertiary amines, in particular with diisopropylethylamine (52%). Some salts with secondary amines could not be isolated in the solid state by the method used. The process of isolation of the present invention generally achieved the best results in the case of salts of Montelukast with primary amines.

Step 4—Crystallization of the Salt of Montelukast with the Primary Amine

The crude salt of Montelukast with the primary amine obtained by the procedure of step 3 was mixed with a suitable solvent; the resulting suspension was stirred and slowly heated up until the formation of a solution, usually up to the boiling point of the solvent. Subsequently, the obtained solution was cooled and stirred, while separation of the crystalline product occurred. The recrystallized product was filtered, washed with a small quantity of the used solvent and dried in vacuo. We have found out that crystallization of salts of Montelukast with primary amines can occur in solvents with various polarities. This is advantageous with regard to the impurities present, the less polar impurities being primarily removed (e.g., eliminate of formula VII, cyclizate of formula VIII) during crystallization from non-polar solvents (e.g. toluene) and the more polar impurities (e.g. the sulfoxide of formula V) being removed during crystallization from polar solvents (e.g. acetonitrile, acetone, ethyl acetate, ethanol or isopropylalcohol). The method of the present invention offered the crystalline product with the chemical purity of 99.5% and higher (HPLC). The chemical purity can also be increased by stirring of the salt of Montelukast with a primary amine in a suitable solvent (e.g. acetonitrile, ethyl acetate, isopropylalcohol). In comparison to crystallization the stirring process provides a higher yield; however, the achieved chemical purity was lower (98.7-99.6% according to HPLC). In this step of the invented process final removal of the impurities primarily soluble in organic solvents occurs. This effect can be achieved by stirring of the crude salt in a suitable solvent or by crystallization of the salt from a supersaturated solution. Increasing of chemical purity of Montelukast in various steps of the process of isolation and crystallization of the salt of Montelukast with iso-propylamine is demonstrated by HPLC chromatograms in FIG. 1.

Step 5—Conversion of the Salts of Montelukast with Primary Amines to the Amorphous Sodium Salt

The crystalline salt of Montelukast with the primary amine obtained by the procedure of step 4 was mixed with a suitable solvent and a solution of a sodium base. The obtained solution of the sodium salt of Montelukast was injected with a syringe or nozzle to an intensively stirred non-polar solvent, while separation of the product in the amorphous form occurred. The resulting product was aspirated, washed with the non-polar solvent used and dried in vacuo. The method of vacuum drying has an extraordinary impact on the resulting content of residual solvents. The drying process used by us is based on using vacuum drying under continuous stream of an inert gas over the dried substance at drying temperatures of up to 50° C. The time course of reduction of the relative weight of the dried sample is demonstrated in FIG. 2. The loss of retained volatiles represents approximately 15% of the original weight in the stream of inert gas mode, while the loss in the mode without the stream of inert solvent under comparable conditions amounted to approximately 11%. The efficiency of the drying process under the inert gas stream is provably higher than the comparable process without any inert gas stream. An advantage of our process of drying of the amorphous form of Montelukast sodium consists in the possibility of obtaining a product with lower-than-limit contents of the solvents used without exposing the substance being dried to the influence of atmospheric oxygen or to the risk of thermal decomposition. Our drying process efficiently removes the residual solvent from the substance, which is difficult to achieve under common conditions. The transformation of the salts of Montelukast with primary amines to the amorphous sodium salt provided the yields of 78-82% and chemical purities of the product 99.5% or higher according to HPLC.

A benefit of the process of isolation of Montelukast of the present invention consists in the use of acetonitrile as the component preventing separation of the salt of Montelukast with a primary amine in a form that is quite unsuitable for large-scale production. The positive influence of acetonitrile has especially been found out in the case of salts of Montelukast with primary amines, preferably in the case of salts with n-propylamine and iso-propylamine. The salts of Montelukast with n-propylamine and iso-propylamine are characterized by advantageous crystallization properties, which are associated with the structure of crystals of the two salts. We have found out that both salts provide stable and mutually very similar crystal forms that can be unambiguously described by means of X-ray Powder Diffraction (XRPD). The present process of purification of Montelukast in the form of its salts with primary amines is beneficial for the ability of these salts to crystallize both from solutions in non-polar solvents (e.g. toluene) and from solutions in polar solvents (e.g. acetonitrile, acetone, ethyl acetate, ethanol, isopropylalcohol). Crystallization from both types of solvents can be combined in order to achieve high chemical purity of the product. A preferable and distinguishing aspect of our process of preparation of the amorphous sodium salt of Montelukast consists in the direct conversion of salts of Montelukast with primary amines to the amorphous sodium salt. So far, either Montelukast acid or salts of Montelukast with secondary amines have been used for the preparation of the amorphous Montelukast sodium. Our solution of preparation of the amorphous form of Montelukast sodium is advantageous in the use of salts of Montelukast with primary amines, which have lower basicity in comparison with secondary amines and sufficient volatility, which allows efficient removal of the primary amine in the preparation of Montelukast sodium without undesired contamination of the product. The invented process of preparation of the amorphous sodium salt of Montelukast is further characterized by the use of a nozzle for introduction of the solution of the sodium salt into the non-polar solvent and by advantageous method of drying of the amorphous sodium salt of Montelukast under reduced pressure and continuous stream of an inert gas. The invented process can be used for the production of Montelukast sodium in a quality required for pharmaceutical substances with a number of benefits.

### BRIEF DESCRIPTION OF DRAWINGS

FIG. 1 shows HPLC chromatograms in various stages of the process of isolation and purification of Montelukast
A HPLC chromatogram of the reaction mixture before the isolation of Montelukast obtained in accordance with Example 1
B HPLC chromatogram of the isolated crude salt of Montelukast with isopropylamine obtained in accordance with Example 2
C HPLC chromatogram of the crystalline salt of Montelukast with isopropylamine obtained in accordance with Example 3
   Sequence of peaks: 1. toluene, 2. alcohol (VI), 3. mesylate (IV), 4. Montelukast (I), 5. eliminate (VII), 6. cyclizate (VIII)

FIG. 2 shows the time course of reduction of the relative weight of the dried sample of the amorphous Montelukast sodium prepared by the procedure of Example 5, dried by the method of Example 11, either under a stream of inert gas (a), or without a stream of inert gas (b).

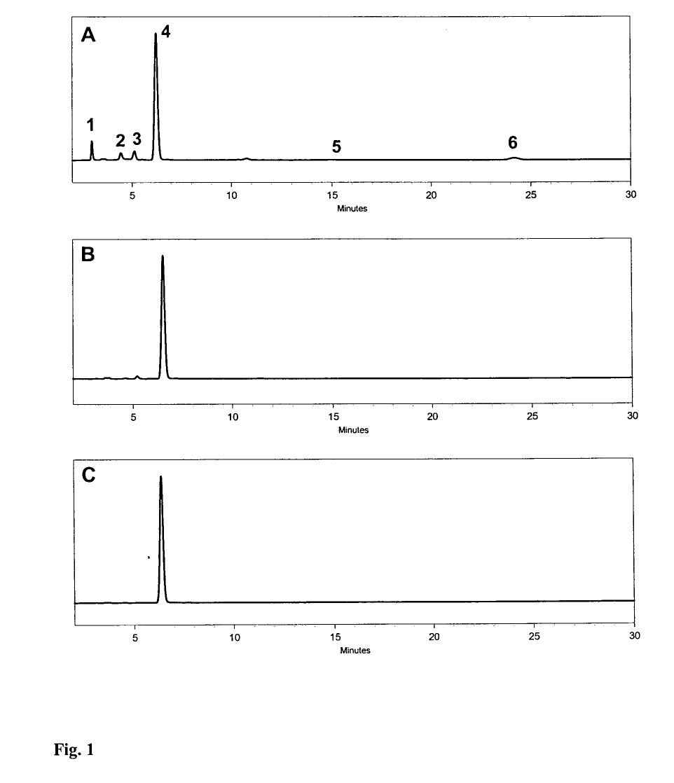

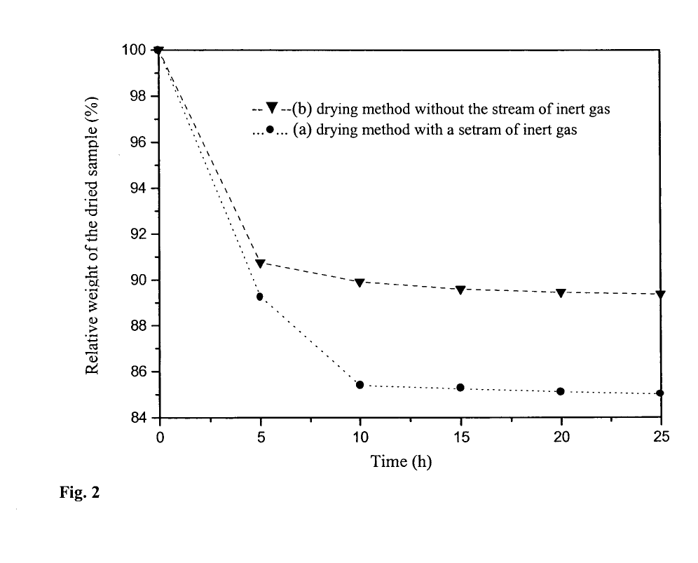

### EXAMPLES

The subject-matter of the invention will be further illustrated by means of the following examples, which however, do not have any influence on the scope of the invention defined in the claims.

### Example 1

### Synthesis, Crude Montelukast Sodium

In 200 ml of toluene, [1-(mercaptomethyl)cyclopropyl]acetic acid (6.62 g), a base (sodium tert-butoxide, 8.50 g) and PEG-600 (26 ml in 30 ml of toluene) were mixed; the mixture was stirred in an argon atmosphere and cooled to ca. −10° C. Then, a solution of 2-(3-(S)-(3-(2-(7-chloroquinolinyl)-ethenyl)phenyl)-3-methanesulfonyloxypropyl)phenyl-2-propanol (26 g) in 120 ml tetrahydrofuran was added to the obtained slurry. The reaction mixture was stirred gradually from −10° C. up to the laboratory temperature for 1 hour. It was further stirred at the laboratory temperature (about 21° C.) for several hours. The reaction mixture was continuously analyzed by means of HPLC: At the end of the monitoring the reaction mixture contained 85.7% of Montelukast.

### Example 2

### Isolation of the Salt of Montelukast with Iso-Propylamine

The reaction mixture from Example 1 was concentrated in vacuo, 100 ml of toluene was added to the residue and again concentrated in vacuo. The residue was diluted with toluene to the volume of 200 ml. It was washed twice with 0.5 M solution of tartaric acid, twice with 100 ml of water and the obtained toluene solution was dried over sodium sulfate. Then, filtration of the drying agent and addition of 50 ml of acetonitrile, 4.5 ml of iso-propylamine and 200 ml of heptane followed. After one hour of stirring another 100 ml of heptane was added to the suspension and it was stirred for another hour. Then, filtration was performed and the cake was washed with 3×50 ml of heptane. After drying at the laboratory temperature in vacuo 19.7 g of an off-white powder were obtained. The yield comprising both the synthesis of the crude sodium salt of Montelukast according to Example 1 and isolation of the salt with iso-propylamine amounted to 75%, HPLC 93.5%.

Using an analogous procedure without the use of acetonitrile the product separated in the form of an oil, which subsequently solidified on the walls of the crystallization vessel. In order to transfer the separated product into the filtration equipment it was necessary to disintegrate it mechanically.

### Example 3

### Crystallization of the Salt of Montelukast with Iso-Propylamine

15.0 g of the salt of Montelukast with iso-propylamine were mixed with 200 ml of toluene and gradually heated up to 95° C. under stirring in an argon atmosphere. Then, being intensively stirred the mixture was slowly cooled down to the laboratory temperature and further stirred for several hours. After that, filtration was performed, the cake was washed with 2×50 ml of heptane. After vacuum drying at the laboratory temperature 12.9 g of an off-white powder was obtained. Crystallization yield 86%, HPLC 99.7%. ¹H NMR (250 MHz, DMSO-D6), δ (ppm) 0.23-0.47 (m, 4H, 2×CH₂ cyclopropyl), 1.08 (d, 6H, 2×CH₃ iso-propyl), 1.44 (s, 6H, 2×CH₃), 2.10-2.30 (m, 4H, 2×CH₂), 2.51 (m, 1H, CH), 2.52 and 2.63 (m, 2H, CH₂), 2.77 and 3.07 (2×m, 2H, CH₂), 3.06 (m, 1H, CH iso-propyl), 4.01 (t, 1H, CH), 5.70 (bb, 4H, NH³⁺, OH), 7.03-8.41 (m, 15H, CH═CH and CH-arom.).

In an analogous procedure the salt of Montelukast with iso-propylamine was crystallized from acetonitrile (1 g dissolved on boiling in 40 ml of the solvent, yield 65%) from acetone (1 g dissolved on boiling in 10 ml of the solvent, yield 46%) from ethyl acetate (1 g dissolved on boiling in 40 ml of the solvent, yield 67%) from ethanol (1 g dissolved at the temperature of 55° C. in 10 ml of the solvent, yield 45%) from isopropylalcohol (1 g dissolved at the temperature of 55° C. in 20 ml of the solvent, yield 70%).

### Example 4

### Separating the Crude Salt of Montelukast with Iso-Propylamine by Stirring in a Solvent

1 g of the salt of Montelukast with iso-propylamine prepared in accordance with Example 2 was mixed with 20 ml of the solvent and the obtained suspension was intensively stirred at the laboratory temperature for 2 hours; then, filtration and vacuum drying was performed. The chemical purity of the salt obtained this way was 98.7-99.5%. Used solvent/yield: acetonitrile (86%), ethyl acetate (81%), isopropyl alcohol (75%).

### Example 5

### Montelukast Sodium-Amorphous

15 ml of toluene were added to 2.11 g of the crystalline salt of Montelukast with iso-propylamine obtained in accordance with Example 3, the suspension was stirred for 20 minutes, then, sodium tert-butoxide (0.34 g) and active charcoal was added and the suspension was further stirred for 45 minutes at the temperature of approximately 35° C. Then, filtration was performed and the clear yellow filtrate was injected to 35 ml of intensively stirred heptane with a syringe. The obtained suspension was further stirred for one hour, after that filtration and vacuum drying was carried out. 1.55 g of powder was obtained. Yield 78%, HPLC 99.6%.

### Example 6

### Synthesis, Crude Montelukast Sodium

In 200 ml of toluene [1-(mercaptomethyl)cyclopropyl]acetic acid (6.72 g), a base (sodium tert-butoxide, 8.50 g) and PEG-600 (26 ml in 30 ml of toluene) were mixed; the mixture was stirred in an argon atmosphere and cooled to ca. −15° C. Then, a solution of 2-(3-(S)-(3-(2-(7-chloroquinolinyl)-ethenyl)phenyl)-3-methanesulfonyloxypropyl)phenyl-2-propanol (26 g) in 120 ml tetrahydrofuran was added to the obtained slurry. The reaction mixture was stirred for 1 hour gradually from −10° C. up to the laboratory temperature. Then, it was further stirred at the laboratory temperature (about 21° C.) for several hours. The reaction mixture was continuously analyzed with HPLC: At the end of the monitoring the reaction mixture contained 82% of Montelukast.

### Example 7

### Isolation of the Salt of Montelukast with N-Propylamine

The reaction mixture from Example 6 was concentrated in vacuo, toluene was added to the residue up to the resulting volume of 200 ml. The obtained solution was washed with 0.5 M solution of tartaric acid, twice with 100 ml of water and dried over sodium sulfate. Then, filtration of the drying agent and the addition of 40 ml of acetonitrile, 4.25 ml of n-propylamine and 200 ml of heptane followed. After one hour of stirring another 100 ml of heptane was added to the suspension and it was stirred for another hour. Then, filtration was performed and the cake was washed with 2×50 ml of heptane. After vacuum drying at the laboratory temperature 17.9 g of an off-white powder were obtained. The yield comprising both the synthesis of the crude sodium salt of Montelukast in accordance with Example 5 and isolation of the salt with n-propylamine amounted to 68%, HPLC 94.3%.

In an analogous procedure without the use of acetonitrile separation of the product occurred in the form of an oil, which subsequently solidified on the walls of the crystallization vessel. For transferring the separated product to the filtration equipment it was first necessary to mechanically disintegrate it.

### Example 8

### Crystallization of the Salt of Montelukast with N-Propylamine

15.0 g of the salt of Montelukast with n-propylamine were mixed with 200 ml of toluene and gradually heated up to 95° C. under the argon atmosphere. Then, being intensively stirred the mixture was slowly cooled down to the laboratory temperature and further stirred for several hours. After that, filtration was performed, the cake was washed with 2×50 ml of heptane. After vacuum drying at the laboratory temperature 11.7 g of an off-white powder was obtained. Crystallization yield 78%, HPLC 99.7%. ¹H NMR (250 MHz, DMSO-D6), δ (ppm) 0.25-0.45 (m, 4H, 2×CH₂-cyclopropyl), 0.85 (t, 3H, CH₃ n-propyl), 1.44 (s, 6H, 2×CH₃), 1.46 (m, 2H, CH₂ n-propyl), 2.10-2.30 (m, 4H, 2×CH₂), 2.49-2.66 (m, 5H, 1×CH₂ n-propyl, 1×CH₂, 1×CH), 2.78 and 3.06 (2×m, 2H, CH₂), 4.01 (t, 1H, CH), 5.89 (bb, 4H, NH³⁺, OH), 7.03-8.41 (m, 15H, CH═CH and CH-arom.).

In an analogous procedure the salt of Montelukast with n-propylamine was crystallized from acetonitrile (1 g dissolved on boiling in 40 ml of the solvent, yield 64%) from acetone (1 g dissolved on boiling in 10 ml of the solvent, yield 51%) from ethyl acetate (1 g dissolved on boiling in 40 ml of the solvent, yield 63%) from ethanol (1 g dissolved at the temperature of 55° C. in 10 ml of the solvent, yield 42%) from isopropylalcohol (1 g dissolved at the temperature of 55° C. in 20 ml of the solvent, yield 69%).

### Example 9

### Separating the Crude Salt of Montelukast with N-Propylamine by Stirring in a Solvent

1 g of the salt of Montelukast with n-propylamine prepared in accordance with Example 7 was mixed with 20 ml of the solvent and the obtained suspension was intensively stirred at the laboratory temperature for 2 hours; then, filtration and vacuum drying was performed. The chemical purity of the salt obtained this way was 98.8-99.6%. Used solvent/yield: acetonitrile (83%), ethyl acetate (78%), isopropyl alcohol (76%).

### Example 10

### Montelukast Sodium-Amorphous

15 ml of toluene were added to 2.11 g of the crystalline salt of Montelukast with n-propylamine obtained in accordance with Example 8, the suspension was stirred for 20 minutes, then, sodium tert-butoxide (0.34 g) was added and the suspension was further stirred at the temperature of approximately 30° C. for 45 minutes. Then, filtration was performed and the clear yellow filtrate was injected to 35 ml of intensively stirred heptane with a syringe. The obtained suspension was further stirred for one hour, after that filtration and vacuum drying was carried out. 1.63 g of a powder was obtained. Yield 82%, HPLC 99.6%.

### Example 11

### Drying of the Amorphous Sodium Salt

In a vacuum drier the amorphous form of Montelukast sodium prepared analogously to Example 5 was dried in two different modes: in the stream of inert gas (a), without any stream of inert gas (b). In both the drying modes identical batches of amorphous sodium Montelukast were dried.

(a) 15 g of the amorphous form of Montelukast sodium was dried in a vacuum drier at the temperature of 50±2° C., pressure of 150±20 mbar and the flow rate of 0.1 m³/h of nitrogen. The drying result is described in Table 1.

TABLE 1
Vacuum drying of Montelukast sodium -
amorphous in a stream of inert gas

| Time (h) | 5 | 10 | 15 | 20 | 25 |
|---|---|---|---|---|---|
| Weight loss on | 10.73 | 14.60 | 14.75 | 14.88 | 14.99 |
| drying (%) | | | | | |

Content of residual toluene at the end of drying/limit (ppm): 44/890
Content of residual heptane at the end of drying/limit (ppm): 390/5000

The content of residual solvents was determined in the standard way by means of gas chromatography.

(b) 15 g of the amorphous form of Montelukast sodium was dried in a vacuum drier at the temperature of 50±2° C., pressure of 150±20 mbar without any nitrogen flow. The drying result is described in Table 2.

TABLE 2
Vacuum drying of Montelukast sodium -
amorphous without any inert gas stream

| Time (h) | 5 | 10 | 15 | 20 | 25 |
|---|---|---|---|---|---|
| Weight loss on | 9.24 | 10.08 | 10.40 | 10.54 | 10.63 |
| drying (%) | | | | | |

### Example 12

### Preparation of Montelukast Salts with Amines from Montelukast Acid

10 ml of toluene, 2.5 ml of acetonitrile and 1.05 of the equivalent quantity of the amine were added to 1 g of Montelukast acid (prepared with the method in accordance with sample 1 from WO 2005/040123 A1). 10 ml of heptane were gradually added to the obtained solution on stirring and the resulting mixture was further stirred. In case of separation of the Montelukast salt with the amine in the solid state the product was filtered, washed with 5 ml of heptane and dried. The results for individual amines are summarized in table 3.

TABLE 3
Preparation and yields of salts of various amines with Montelukast

| | | | Melting | |
|---|---|---|---|---|
| | Separated | Yield | point | Start of |
| Amine | form | (%) | (° C.) | crystallization |
| n-propylamine | solid state | 95 | 97-100 | 10 min |
| iso-propylamine | solid state | 94 | 97-100 | 10 min |
| t-butylamine | solid state | 92 | 98-101 | 45 min |
| benzylamine | solid state | 85 | 93-96 | 2 h |
| α-methylbenzylamine | solid state | 92 | 98-102 | 5 min |
| 2-methylaminoethanol | oil | — | — | — |
| dipropylamine | not separated | — | — | — |
| diisopropylamine | not separated | — | — | — |
| dicyclohexylamine | solid state | 82 | 118-121 | 2 h |
| diisopropylethylamine | solid state | 52 | 152-154 | 3 h |

ANALYTIC METHODS AND DATA (A, B, C, D): Conversion and selectivity within our process of preparing Montelukast as well as the quality of salts of Monelukast with primary amines and sodium Montelukast were determined by means of the HPLC method. Analytic data obtained by means of X-Ray Powder Diffraction (XRPD) unambiguously characterize the crystalline salts of Montelukast with n-propylamine and iso-propylamine. The chemical structure of Montelukast salts with amines was checked by means of ¹H NMR, and the melting points of the salts of Montelukast with amines obtained in the solid state were also measured.

### A High Performance Liquid Chromatography (HPLC)

HPLC chromatograms were measured with the EliteLachrom device made by the Hitachi Company. A column filled with the stationary phase RP-18e was used for the analyses. As the mobile phase a mixture of acetonitrile (80%) and 0.1M aqueous solution of ammonium formate adjusted to pH 3.6 with formic acid (20%) was used. The measurements were carried out in the isocratic mode with the flow rate of the mobile phase 1.5 ml/min.

### B X-Ray Powder Diffraction (XRPD)

XRPD diffraction patterns of the crystalline salts of Montelukast with n-propylamine and iso-propylamine that were prepared in accordance with Examples 3 and 8 were measured with the X'PERT PRO MPD PANalytical diffractometer under the following experimental conditions:
Radiation: CuKa (λ=1.54178 Å)
Monochromator: graphite
Excitation voltage: 45 kV
Anode current: 40 mA
Measured range: 2-40° 2θ
Increment: 0.01 2θ

The values of measured characteristic diffraction angles 20, interplanar distances d and relative signal intensities are summarized in Tables 4 and 5.

TABLE 4
Values of characteristic diffraction angles 2θ, interplanar
distances d and relative signal intensities in XRPD records
of the crystalline salt of Montelukast with n-propylamine.

| (° 2θ) | d (Å) | Iᵣₑₗ (%) |
|---|---|---|
| 3.35 | 26.332 | 6.34 |
| 8.85 | 9.983 | 100.00 |
| 9.92 | 8.906 | 19.86 |
| 16.68 | 5.310 | 17.93 |
| 17.41 | 5.090 | 74.40 |
| 18.41 | 4.814 | 28.16 |
| 19.36 | 4.580 | 47.13 |
| 20.15 | 4.404 | 40.44 |
| 20.58 | 4.311 | 37.94 |
| 22.98 | 3.867 | 30.00 |
| 23.70 | 3.752 | 60.30 |
| 26.16 | 3.403 | 35.50 |

TABLE 5
Values of characteristic diffraction angles 2θ, interplanar
distances d and relative signal intensities in XRPD records
of the crystalline salt of Montelukast with iso-propylamine.

| (° 2θ) | d (Å) | Iᵣₑₗ (%) |
|---|---|---|
| 3.36 | 26.305 | 10.96 |
| 9.08 | 9.733 | 100.00 |
| 9.83 | 8.986 | 20.64 |
| 16.46 | 5.383 | 17.40 |
| 17.17 | 5.159 | 81.54 |
| 18.16 | 4.880 | 28.86 |
| 19.06 | 4.652 | 33.13 |
| 20.22 | 4.388 | 50.33 |
| 20.57 | 4.315 | 60.69 |
| 22.86 | 3.888 | 25.18 |
| 23.88 | 3.724 | 47.77 |
| 26.25 | 3.392 | 25.33 |

### C ¹H NMR

¹H NMR spectra of the salts of Montelukast with amines were measured with the Avance 250 Bruker spectrometer with the measuring frequency of 250.13 MHz. The spectra were measured for solutions in DMSO-D6, chemical shifts were related to the internal standard TMS δ=0 ppm.

### D Melting Point

Melting points of the salts of Montelukast with amines prepared in accordance with Example 10 were measured on the Kofler block with the sample heating speed of 10° C. (to 70° C.) and 4° C. (over 70° C.) per minute. The measured values of melting points are summarized in Table 3.

1. A method for isolation of Montelukast of formula I and its pharmaceutically acceptable salts, wherein a solution of the crude salt of Montelukast with an alkali metal is transformed to a solution of Montelukast acid, followed by releasing and isolation of the crystalline salt of Montelukast with a primary amine in the environment of at least one solvent and Acetonitrile; performing recrystallization of the salt of Montelukast with the primary amine with simultaneous removal of chemical impurities and using the salt of Montelukast with the primary amine for conversion into a form of Montelukast sodium of formula II.

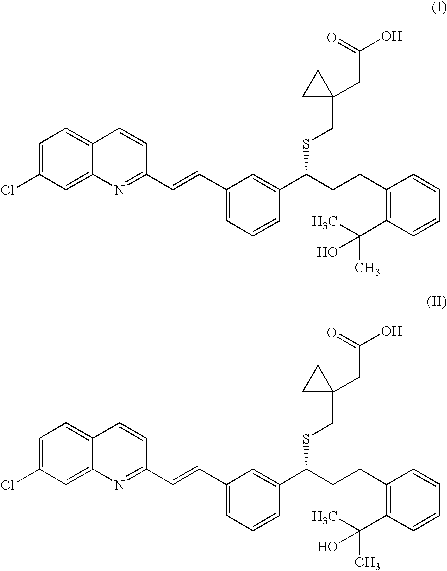

2. The method according to claim 1, comprising the following steps:
Conversion of the mixture containing the salt of Montelukast with an alkali metal to a solution of Montelukast acid;
Isolation of the salt of Montelukast with the primary amine using at least one solvent and acetonitrile;
Crystallization of the salt of Montelukast with the primary amine from at least one solvent or separation of the salt of Montelukast with the primary amine by stirring with at least one solvent; and
Conversion of salts of Montelukast with primary amines to an amorphous sodium salt.

3. The method according to claim 1, wherein an aqueous solution of an acid is used for the conversion of the solution of the crude salt of Montelukast with an alkali metal to the solution of Montelukast acid.

4. The method according to claim 1, wherein a solution of a carboxylic acid is used for the conversion of the solution of the crude salt of Montelukast with an alkali metal to the solution of Montelukast acid.

5. The method according to claim 4, wherein a solution of a carboxylic acid selected from the group comprising acetic acid, formic acid, succinic acid, maleic acid, fumaric acid and tartaric acid is used.

6. The method according to claim 1, wherein a crystalline salt of Montelukast with a primary amine is used for the isolation of Montelukast.

7. The method according to claim 6, wherein a primary amine selected from the group comprising methylamine, ethylamine, propylamine, isopropylamine, butylamine, -methylbenzylamine and benzylamine is used for the crystalline salt of Montelukast.

8. The method according to claim 1, wherein at least one solvent, selected from the group comprising benzene, toluene, xylenes, tetrahydrofuran, diethylether, acetone, methylethylketone, dimethyl carbonate, ethyl acetate, cyclohexane, hexane, heptane, pentane and petroleum ether, is used for the isolation of the salt of Montelukast with the primary amine.

9. The method according to claim 1, wherein acetonitrile is used for the isolation of the crystalline salt of Montelukast with the primary amine.

10. The method according to claim 1, wherein at least one solvent, selected from group comprising benzene, toluene, xylenes, tetrahydrofuran, diethylether, acetone, methylethylketone, dimethyl carbonate, ethanol, isopropyl alcohol, cyclohexane, hexane, heptane, pentane and petroleum ether, is used for the crystallization of the salt of Montelukast with the primary amine.

11. (canceled)

12. (canceled)

13. The method according to with claim 1, wherein a base which contains sodium ions is used for the conversion of the salt of Montelukast with the primary amine to the pharmaceutically useful amorphous sodium salt.

14. (canceled)

15. (canceled)

16. The method according to claim 1, wherein at least one solvent selected from the group comprising benzene, toluene, xylenes, tetrahydrofuran, diethylether, acetone, methylethylketone, acetonitrile, dimethyl carbonate, ethyl acetate, methanol, ethanol, isopropyl alcohol, cyclohexane, hexane, heptane, pentane and petroleum ether is used for the conversion of the salt of Montelukast with the primary amine to the pharmaceutically useful amorphous sodium salt.

17. The method according to claim 1, wherein the solution of the sodium salt of Montelukast in a suitable solvent is injected with a nozzle to a stirred non-polar solvent for the conversion of the salt of Montelukast with the primary amine to the pharmaceutically useful amorphous sodium salt.

18.-27. (canceled)
